(19) United States
(12) Patent Application Publication
(10) Pub. No.: US 2023/0279390 A1
(43) Pub. Date: Sep. 7, 2023

(54) ORTHOGONALLY LINKED MULTIMERIC OLIGONUCLEOTIDES

(71) Applicant: MPEG LA, L.L.C., Chevy Chase, MD (US)

(72) Inventor: Jonathan Miles Brown, Silver Spring, MD (US)

(21) Appl. No.: 17/925,807

(22) PCT Filed: May 18, 2021
(86) PCT No.: PCT/US2021/033028

§ 371 Date: Nov. 16, 2022

Related U.S. Application Data
(60) Provisional application No. 63/026,929, filed on May 19, 2020.
(60) Provisional application No. 63/093,097, filed on Oct. 16, 2020.

(51) Int. Cl. C12N 15/11

(52) U.S. Cl. CPC C12N 15/111; C12N 2310/11; C12N 2310/14; C12N 2310/51; C12N 2310/351; C12N 2310/3519; C12N 2330/30

(57) ABSTRACT

Orthogonally linked multi-conjugates (such as multimeric oligonucleotides) are disclosed, along with methods of synthesizing them using orthogonal linking strategies to join together subunits that are biological moieties.

### INCORPORATION BY REFERENCE TO ANY PRIORITY APPLICATIONS

Any and all applications for which a foreign or domestic priority claim is identified in the PCT Request as filed with the present application are hereby incorporated by reference.

### FIELD OF THE DISCLOSURE

The present disclosure relates to multimeric oligonucleotides. More specifically, the present disclosure relates to orthogonally linked multimeric oligonucleotides, methods of synthesizing multimeric oligonucleotides using orthogonal linking strategies, and methods of using the resulting oligonucleotides.

### BACKGROUND

Oligonucleotides are now a well-established class of therapeutics with multiple applications and ongoing clinical trials. However, many factors still limit the development and use of oligonucleotide therapeutics, for example, the delivery of the oligonucleotide to a target cell and the subsequent internalization of the oligonucleotide into the target cell in sufficient quantities to achieve a desired therapeutic effect.

To address this issue, oligonucleotides conjugated to ligands targeting specific cell surface receptors have been investigated. The use of one such ligand, N-acetylgalactosamine (GalNAc), has become a method of choice for oligonucleotide delivery to hepatocytes due to its highly specific and efficient binding to the asialoglycoprotein receptor, which is expressed in large numbers on the surface of these cells.

However even with the use of GalNAc-conjugated oligonucleotides, a high proportion of the compound is lost via excretion through the kidney. To counter this, multimers of oligonucleotides have been prepared wherein individual oligonucleotide subunits have been linked together via covalently bonded intermediates or “linkers”. These linkers have been introduced on the synthesizer or in aqueous solution after synthesis, deprotection and purification of the oligonucleotide.

A variety of linkers have been employed, including ones that are stable under in vivo conditions and others that are cleaved inside the target cell thereby liberating the individual oligonucleotide subunits. The most common type of cleavable linkers used have been short sequences of single-stranded unprotected nucleotides such as dTdTdTdT and dCdA, which are cleaved by intracellular nucleases, and disulfide-based linkers which are cleaved by the reductive environment inside the cell.

Another technique that has been successfully employed in the synthesis of multimeric oligonucleotides is asymmetric annealing whereby a single-stranded oligonucleotide bonded via a linker to another oligonucleotide is annealed to a complementary single-stranded oligonucleotide, optionally also bonded via a linker to another oligonucleotide, these steps being repeated until a multimer of the desired length is obtained.

Both homo- and hetero-multimers have been prepared via these methods and multimers in the 4-mer to 8-mer range exhibit notably enhanced serum half-lives and bioactivities.

However, these methods have limitations. Nuclease cleavable linkers can only be introduced via the synthesizer. Disulfide linkages can be introduced both on the synthesizer and in aqueous solution after purification of the precursor. However, it is not possible to maintain an internal disulfide group in a multimer while simultaneously reducing a terminal disulfide to a thiol for subsequent linking reactions. Finally, the asymmetric annealing method is difficult to apply to homo-multimers as random polymerization may occur.

There is therfore a need for additional methods and materials to act as linkers in the assembly and synthesis of multimeric oligonucleotides.

### SUMMARY OF THE DISCLOSURE

The present disclosure relates to orthogonally linked multi-conjugates of biological moieties and methods of synthesizing them using orthogonal linking strategies. The disclosure is applicable to all types of biological moieties, including but not limited to proteins, oligopeptides and oligonucleotides, double-stranded and single-stranded, including for example, siRNAs, saRNAs, miRNAs, aptamers, and antisense oligonucleotides. Strategies described herein as being applicable to multi-conjugates of oligonucleotides (multimeric oligonucleotides) will be understood as being generally applicable to multi-conjugates of other biological moieties, and vice versa, unless the context clearly indicates otherwise.

The present disclosure provides methods for the synthesis of a multi-conjugate, such as a multimeric oligonucleotide (“multimer”) comprised of two or more oligonucleotides ( “subunits”; each individually a “subunit”) linked together via covalent linkers, wherein the subunits may be multiple copies of the same subunit or differing subunits.

The present disclosure also relates to new synthetic intermediates and methods of synthesizing the multimeric oligonucleotides using the synthetic intermediates.

The present disclosure also relates to methods of using the multimeric oligonucleotides, for example in modulating gene expression, biological research, treating or preventing medical conditions, and/or to produce new or altered phenotypes.

In an embodiment, the disclosure provides a multimeric oligonucleotide comprising subunits ********, wherein each of the subunits ******** is independently a single or double stranded oligonucleotide, and one or more of the subunits ******** is joined to another subunit by a covalent linker •, and wherein two or more subunits comprise different thiol groups at either the 5′ or 3′ end.

In an embodiment, at least one subunit ******** comprises at least one partial single-stranded oligonucleotide annealed to a complementary strand. For example, in an embodiment, at least one subunit ******** comprises two partial single-stranded oligonucleotides annealed to a complementary strand.

In an embodiment, the disclosure provides a multimeric oligonucleotide wherein a first subunit ******** comprises a 3′ or 5′ reactive thiol group and a second subunit ******** comprises a 3′ or 5′ protected thiol group.

In an embodiment, the disclosure provides a process for preparing a multimeric oligonucleotide, comprising:

providing a first subunit reactant, the first subunit reactant comprising a 3′ or 5′ reactive thiol group and, optionally, a 5′ or 3′ ligand, providing a second subunit reactant comprising a 3′ or 5′ protected thiol group and a 5′ or 3′ group, the 5′ or 3′ group being reactive with the reactive thiol group on the first subunit reactant; and intermixing the first subunit reactant with the second subunit reactant under reaction conditions selected to react the 3′ or 5′ reactive thiol group of the first subunit reactant with the 5′ or 3′ group of the second subunit reactant to thereby form a covalent bond linking the first subunit to the second unit. In an embodiment, the 5′ or 3′ group of the second subunit reactant is an electrophilic group such as a maleimide group. In an embodiment, the optional 5′ or 3′ ligand is a chemical or biological moiety L as described elsewhere herein with respect to Structure 1.

In an embodiment, the disclosure provides a multimeric oligonucleotide wherein the conditions for the removal of the thiol protecting group do not affect the stability of the covalent linkers •.

In an embodiment, the disclosure provides a multimeric oligonucleotide wherein the thiol is protected as an alkyl, alkoxy, benzyl or aryl thioether.

In an embodiment, the disclosure provides a multimeric oligonucleotide wherein the thiol is protected as an alkyl silylthioether.

In an embodiment, the disclosure provides a multimeric oligonucleotide wherein the thiol is protected as alkyl or aryl thioester.

In an embodiment at least two subunits ******** are substantially different. In an embodiment, all of the subunits are substantially different.

In an embodiment, at least two subunits ******** are substantially the same or are identical. In an embodiment, all of the subunits ******** are substantially the same or are identical.

In an embodiment, each nucleic acid strand within a subunit is independently 5-30, 10-30, 17-27, 19-26, or 20-25 nucleotides in length.

In an embodiment, one or more subunits are double-stranded. In an embodiment, one or more subunits are single-stranded.

In an embodiment, the subunits comprise a combination of single-stranded and double-stranded oligonucleotides.

In an embodiment, one or more nucleotides within an oligonucleotide is an RNA, a DNA, or an artificial or non-natural nucleic acid analog.

In an embodiment, at least one of the subunits comprises RNA.

In an embodiment, at least one of the subunits comprises an siRNA, an saRNA, or a miRNA.

In an embodiment, at least one of the subunits comprises an antisense oligonucleotide.

In an embodiment, at least one of the subunits comprises a double-stranded siRNA.

In an embodiment, two or more siRNA subunits are joined by covalent linkers attached to the sense strands of the siRNA.

In an embodiment, one or more of the covalent linkers • comprise a cleavable covalent linker.

In an embodiment, the cleavable covalent linker contains an acid cleavable bond, a reductant cleavable bond, a bio-cleavable bond, or an enzyme cleavable bond.

In an embodiment, the disclosure provides a multi-conjugate comprising a plurality of subunits ******** joined to one another by one or more covalent linkers •, wherein the multi-conjugate comprises Structure 4:

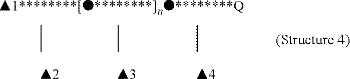

wherein:
each of the subunits ********, independently, is a biological moiety;
at least one covalent linker • is a sulfur-containing covalent linker;
each of ▲₁, ▲₂, ▲₃, and ▲₄ is a group that is independently absent or comprises a functional moiety joined to a subunit and, optionally, a spacer group joining the functional moiety to the subunit;
Q is a group that comprises a sulfur-containing end group, e.g., a protected thiol group; and
n is an integer, e.g., 0, 1, 2, 3, 4, 5, 6, 7, or 8.

In an embodiment, at least one of the subunits ******** present in Structure 4. is not a nucleic acid. In an embodiment, at least one of the subunits ******** present in Structure 4 comprises an oligopeptide or a protein.

In an embodiment, at least one of the functional moieties ▲₁, ▲₂, ▲₃, and ▲₄ is present in in Structure 4. For example, in an embodiment, the at least one functional moiety that is present is a targeting ligand. In another embodiment, the at least one functional moiety that is present is a detectable label (e.g., a dye).

In an embodiment, the disclosure provides a multimeric oligonucleotide comprising a plurality of subunits ******** and a sulfur-containing end group; wherein each of the subunits ******** is independently a single or double stranded oligonucleotide; and two or more of the subunits ******** are joined to one another by a sulfur-containing covalent linker ◊.

The term “sulfur-containing end group” as used herein refers to a chemical moiety that (1) contains a sulfur that is not attached to another sulfur and (2) is attached to an end of a multi-conjugate, e.g., the 3′ or 5′ end of the multimeric oligonucleotide. Thus, the sulfur-containing end group is not a disulfide. For example, in various embodiments the sulfur-containing end group is a thiol group or a protected thiol group.

In an embodiment, the sulfur-containing end group (e.g., the end group Q in Structure 4) comprises a protected thiol group that is deprotectable under a deprotection condition; and the sulfur-containing covalent linker ◊ is stable under the deprotection condition.

In an embodiment, the sulfur-containing covalent linker ◊ comprises a sulfur-containing cleavable group, including but not limited to C2-C10 alkyldithio, thioether, thiopropionate, or disulfide. In an embodiment, the sulfur-containing covalent linker ◊ is cleavable under a cleavage condition that is not the deprotection condition. In an embodiment, the sulfur-containing covalent linker ◊ comprises a sulfur-containing cleavable group that is cleavable under a cleavage condition that is not the deprotection condition.

In an embodiment, the sulfur-containing end group is a protected thiol group.

In an embodiment, the group ▲₁ comprises a moiety of the formula L-R¹, wherein L is a functional moiety and R¹ is a spacer group joining R¹ to the subunit ********

In an embodiment, a multimeric oligonucleotide as described herein is represented by the following Structure 1:

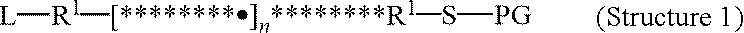

wherein each of the subunits ******** is independently a single or double stranded oligonucleotide; each • is a covalent linker, of which at least one is a sulfur-containing covalent linker ◊; n is an integer in the range of 1 to 9; L is a moiety that may be present or absent and has biological activity or affinity; each R¹ is individually a spacer group that may be present or absent; and S-PG is a protected thiol group.

In an embodiment, n in Structure 1 is an integer in the range of 2 to 6.

In an embodiment, L in Structure 1 and any of ▲₁, ▲₂, ▲₃, and ▲₄ in Structure 4 comprises a targeting ligand. Examples of ligands that can be targeting ligands include antibody, antibody fragment, double chain antibody fragment, single chain antibody fragment; other proteins, for example, a glycoprotein (e.g., transferrin) or a growth factor; peptide (e.g., the RGD ligand or gastrin-releasing peptides); nucleic acid (e.g., an aptamer), a peptide or peptide derivative (e.g., DUPA); a natural or synthetic carbohydrate, for example, a monosaccharide (e.g., galactose, mannose, N-Acetylgalactosamine [“GalNAc”]), polysaccharide, or a cluster such as lectin binding oligo saccharide, diantennary GalNAc, or triantennary GalNAc; a lipid, for example, a sterol (e.g., cholesterol), phospholipid (e.g., phospholipid ether, phosphatidylcholine, lecithin); a vitamin compound (e.g., tocopherol or folate); immunostimulant (e.g., a CpG oligonucleotide); an amino acid; an element (e.g., gold); or a synthetic small molecule (e.g., anisamide or polyethylene glycol). For example, in various embodiments L comprises an aptamer, N-Acetylgalactosamine (GalNAc), folate, lipid, cholesterol, or transferrin.

In an embodiment, L in Structure 1 and any of ▲₁, ▲₂, ▲₃, and ▲₄ in Structure 4 comprises an endosomal escape moiety. For example, in various embodiments the endosomal escape moiety comprises a membrane disrupting, altering, or destabilizing peptide, lipid, polymer, or small molecule.

In an embodiment, L in Structures 1 and any of ▲₁, ▲₂, ▲₃, and ▲₄ in Structure 4 comprises a chemical or biological moiety, including, e.g., a biologically active moiety having biological activity or affinity. A biologically active moiety is any molecule or agent that has a biological effect, preferably a measurable biological effect. Chemical or biological moieties include, e.g., proteins, peptides, amino acids, nucleic acids, targeting ligands, carbohydrates, polysaccharides, lipids, organic compounds, and inorganic chemical compounds.

In an embodiment of a multi-conjugate or multimeric oligonucleotide as described herein, at least one subunit ******** comprises Structure 2:

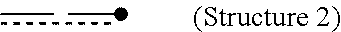

wherein: each

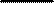

is a partial single-stranded oligonucleotide;
● is a covalent linker joined to a partial single-stranded oligonucleotide; and 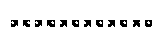 is a complementary strand annealed to the partial single-stranded oligonucleotides.

For example, an embodiment of a multi-conjugate or multimeric oligonucleotide as described herein comprises Structure 3:

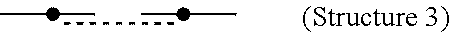

wherein: each 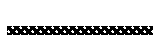 is a single-stranded oligonucleotide; each 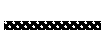 is a partial single-stranded oligonucleotide; each ● is a covalent linker joined to a partial single-stranded oligonucleotide; and ............ is a complementary strand annealed to the partial single-stranded oligonucleotides.

In an embodiment, the at least one covalent linker ● of a multi-conjugate (e.g., a multimeric oligonucleotide) as described herein comprises Structure 5:

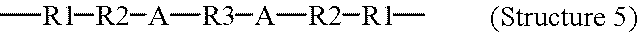

wherein:
each R1 is independently a group comprising phosphodiester, thiophosphodiester, sulfate, amide, triazole, heteroaryl, ester, ether, thioether, disulfide, thiopropionate, acetal, glycol, or is absent;
each R2 is independently a spacer group, or is absent;
each A is the same and is a group comprising the reaction product of a nucleophile and an electrophile; and
R3 is a group comprising a C₂-C₁₀ alkyl, C₂-C₁₀ alkoxy, C₁-C₁₀ aryl, amide, C₂-C₁₀ alkyldithio, ether, thioether, ester, oligonucleotide, oligopeptide, thiopropionate, or disulfide.

In an embodiment, Structure 5 comprises a sulfur-containing covalent linker ◊, wherein R3 is a group comprising C₂-C₁₀ alkyldithio, thioether, thiopropionate, or disulfide.

In an embodiment, at least one covalent linker ● of a multi-conjugate (e.g., a multimeric oligonucleotide) as described herein does not comprise a sulfur-containing covalent linker 0.

In an embodiment of Structure 5, each R¹ is independently a group comprising phosphodiester or thiophosphodiester. In another embodiment, each R1 is independently a group comprising a heteroaryl. In an embodiment, the heteroaryl contains 1, 2, 3, or 4 ring nitrogen atoms and 1, 2, 3, 4, 5, 6, 7, 8 or 9 ring carbon atoms.

In another embodiment of Structure 5, each R² independently comprises a C₂-C₁₀ alkyl, C₂-C₁₀ alkoxy, or C₁-C₁₀ aryl, or is absent. In an embodiment, the C₁-C₁₀ aryl is a C₅₋₆ aryl, such as phenyl or pyridinyl. In an embodiment, the C₁-C₁₀ aryl is a heteroaryl that contains 1, 2, 3, or 4 ring nitrogen atoms and 1, 2, 3, 4, 5, 6, 7, 8 or 9 ring carbon atoms.

In another embodiment of Structure 5, the nucleophile and electrophile of A comprise (i) a thiol and a maleimide, optionally wherein the reaction product of the thiol and maleimide is a derivative of succinamic acid; (ii) a thiol and a vinylsulfone; (iii) a thiol and a pyridyldisulfide; (iv) a thiol and an iodoacetamide; (v) a thiol and an acrylate; (vi) an azide and an alkyne; or (vii) an amine and a carboxyl.

In another embodiment of Structure 5, A is a group comprising the reaction product of a thiol and a maleimide, optionally wherein the reaction product of the thiol and maleimide is a derivative of succinamic acid.

In another embodiment of Structure 5, R³ is a group comprising a thiopropionate or disulfide. In another embodiment of Structure 5, each R² independently comprises a C₂-C₁₀ alkyl, C₂-C₁₀alkoxy, or C₁-C₁₀aryl, or is absent. In an embodiment, the C₁-C₁₀aryl is a C₅₋₆ aryl, such as phenyl or pyridinyl. In an embodiment, the C₁-C₁₀ aryl is a heteroaryl that contains 1, 2, 3, or 4 ring nitrogen atoms and 1, 2, 3, 4, 5, 6, 7, 8 or 9 ring carbon atoms.

In an embodiment, the sulfur-containing covalent linker ◊ comprises a linkage represented by -R¹-R²-R¹-, wherein each R¹ is individually absent or a spacer group; and wherein R² is a thiopropionate or disulfide group.

In an embodiment, the sulfur-containing end group or protected thiol group does not comprise a thiopropionate group or a disulfide group.

In an embodiment, at least one R¹ in the -R¹-R²-R¹- linkage is a spacer group that comprises a group selected from C₁₋₁₀ alkyl, C₁₋₁₀ alkoxy, 5-10 membered aryl, 5-10 membered heteroaryl, 5-10 membered heterocyclyl, -(C₁₋₁₀ alkyl)-(5-10 membered aryl)-, -(C₁₋₁₀ alkyl)-(5-10 membered heteroaryl)-, and -(C₁₋₁₀ alkyl)-(5-10 membered heterocyclyl)-.

In an embodiment, at least one R¹ in the -R¹-R²-R¹- linkage is a spacer group that comprises a phosphorus-containing linkage. Examples of phosphorus-containing linkages include, but are not limited to, phosphorothioates, enantiomerically enriched phosphorothioates, phosphorodithioates, phosphotriesters, aminoalkylphosphotriesters, methyl and other alkyl phosphonates comprising 3′alkylene phosphonates and enantiomerically enriched phosphonates, phosphinates, phosphoramidates comprising 3′- amino phosphoramidate and aminoalkylphosphoramidates, thionophosphoramidates, thilotioalkylphosphonates, thionoalkylphosphotriesters, and boranophosphates having normal 3 ‘-5′ linkages, 2′ -5′ linked analogs of these, and those having inverted polarity wherein the adjacent pairs of nucleoside units are linked 3′-5′ to 5′-3′ or 2′-5′ to 5′-2′.

In an embodiment, at least one R¹ in the -R¹-R²-R¹- linkage is a spacer group that comprises a phosphate linking group, a thiophosphate linking group, a phosphonate linking group, or a dithiophosphate linking group.

In an embodiment, at least one R¹ in the -R¹-R²-R¹- linkage is a spacer group that comprises a C₁₋₆ alkyl.

In an embodiment, at least one R¹ in the -R¹-R²-R¹- linkage is a spacer group that comprises a linking group represented by

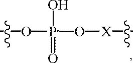

wherein each X independently comprises alkyl, alkyl ether, ester, aryl, heteroaryl, heterocyclyl, alkyl-aryl, alkyl-heteroaryl, or alkyl-heterocyclyl. .

In an embodiment, at least one R¹ in the -R¹-R²-R¹- linkage is a spacer group that further comprises a pyrrolidinyl-2,5-dione.

In an embodiment, the linkage represented by-R¹-R²-R¹- is also represented by:

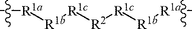

wherein: each R^{1a} is independently absent, or is present and is

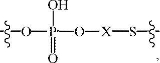

or

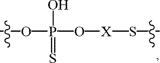

where m is an integer in the range of 1 to 10; and each X independently comprises alkyl, alkyl ether, ester, aryl, heteroaryl, heterocyclyl, alkyl-aryl, alkyl-heteroaryl, or alkyl-heterocyclyl; each R^{1b} is independently absent, or is present and is

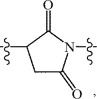

or

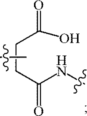

each R^{1c} is independently C₁₋₁₀ alkyl or C₁₋₁₀ alkoxy, and R² is a thiopropionate or disulfide group.

In an embodiment, the linkage represented by -R¹-R²-R¹- comprises

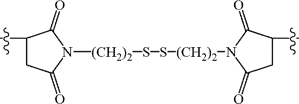

or a ring-opened derivative thereof.

In an embodiment, the linkage represented by -R¹-R²-R¹- is:

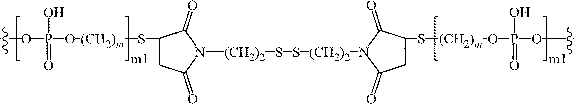

or a ring-opened derivative thereof, wherein each in and m1 are individually an integer in the range of 1 to 10, such as 1, 2, or 3.

In an embodiment, the sulfur-containing end group (e.g., the end group Q in Structure 4) is a protected thiol group of the formula S-PG, where PG is a protecting group that is deprotectable under a deprotection condition to form a thiol group. Various protecting groups are known to those skilled in the art as informed by the present disclosure.

In an embodiment, the sulfur-containing end group (e.g., the end group Q in Structure 4) is a protected thiol group of the formula S-PG that comprises a protecting group PG selected from optionally substituted alkyl, optionally substituted alkylalkoxy, optionally substituted trialkylsilyl, optionally substituted arylalkylsilyl, optionally substituted aryl, optionally substituted benzyl, optionally substituted acyl and optionally substituted benzoyl. For example, in various embodiments the sulfur-containing end group is a protected thiol group of the formula S-PG that comprises a protecting group PG selected from trityl, methoxytrityl, dimethoxytrityl, methylmethoxy, triisopropylsilyl, dinitrophenyl, nitrophenyl, acetyl and benaoyl.

In an embodiment, at least two subunits ******** are substantially different from one another. For example, in some embodiments, the two substantially different subunits have a sequence homology of 90% or less. In some embodiments, the two substantially different subunits are not identical. In some embodiments, the two substantially different subunits have different biological activity. In some embodiments, the two substantially different subunits have different patterns of chemical modification. In some embodiments, the two substantially different subunits differ from one another in two or more of the aforementioned ways.

In an embodiment, the multi-conjugate (e.g., multimeric oligonucleotide) comprises two, three, four, five, or six subunits ********

In an embodiment, one or more subunits ******** are an oligonucleotide.

In an embodiment, one or more subunits ******** are an oligopeptide or a protein.

In an embodiment, each nucleic acid strand within a subunit is 5-30, 15-30, 17-27, 19-26, or 20-25 nucleotides in length.

In an embodiment, one or more subunits ******** are a double-stranded RNA.

In an embodiment, one or more subunits ******** are a single-stranded RNA.

In an embodiment, the subunits ******** comprise a combination of single-stranded and double-stranded oligonucleotides.

In an embodiment, each subunit ******** is an RNA, a DNA, or an artificial or non-natural nucleic acid analog thereof.

In an embodiment, each subunit ******** is an siRNA, an saRNA, or a miRNA.

In an embodiment, each subunit ******** is a double-stranded siRNA.

In an embodiment, a multi-conjugate (e.g. a multimeric oligonucleotide) as described herein comprises a cleavable covalent linker CL joining two or more of the subunits ********, the cleavable covalent linker CL being different from the covalent linker •.

In an embodiment, the cleavable covalent linker CL comprises an acid cleavable bond, a reductant cleavable bond, a bio-cleavable bond, or an enzyme cleavable bond.

In an embodiment, the cleavable covalent linker CL is cleavable under intracellular conditions.

In an embodiment, the disclosure provides a process for preparing a multimeric oligonucleotide of Structure 1d, comprising deprotecting a compound of Structure 1a to form a compound of Structure 1b; and reacting the compound of Structure 1b with a compound of Structure 1c under conditions selected to form a compound of Structure 1d, as follows:

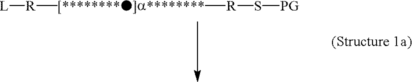

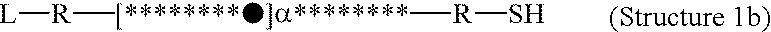

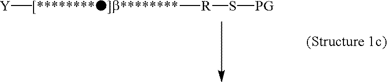

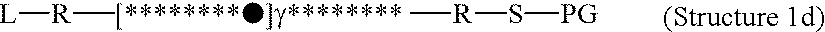

Wherein L is a bioactive moiety that may be present or absent; each R is individually a spacer group that may be present or absent; each ******** is independently a single or double stranded oligonucleotide; each ● is a covalent linker joining adjacent oligonucleotide subunits; S-PG is a protected sulfur-containing end group, optionally a protected thiol group, that is deprotectable under a deprotection condition; Y is a reactive group selected to react with the -R-SH group of Structure 1b to form one of the covalent linkers ● of Structure 1d; γ is an integer in the range of 1to 9; α and β are each individually an integer in the range of 0 to 8, selected such that α + β + 1 = γ; and at least one ● is a sulfur-containing covalent linker ◊ that is stable under the deprotection condition; optionally the sulfur-containing covalent linker comprises a sulfur-containing cleavable group.

In an embodiment, the disclosure provides a process for preparing a multimeric oligonucleotide of Structure If comprising deprotecting a compound of Structure 1a to form a compound of Structure 1 b; and reacting the compound of Structure 1b with a compound of Structure 1e under conditions selected to form a compound of Structure 1f, as follows:

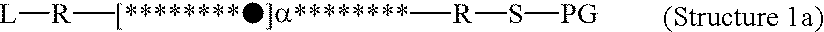

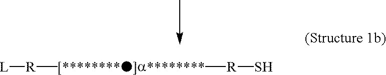

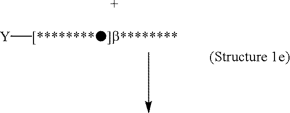

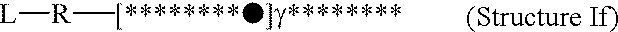

Wherein L is a moiety that may be present or absent and has biological activity or affinity; each R is individually a spacer group that may be present or absent; each ******** is independently a single or double stranded oligonucleotide; each ● is a covalent linker joining adjacent oligonucleotide subunits; S-PG is a protected sulfur-containing end group, optionally a protected thiol group, that is deprotectable under a deprotection condition; Y is a reactive group selected to react with the -R-SH group of Structure 1b to form one of the covalent linkers ● of Structure If, γ is an integer in the range of 1 to 9; α and β are each individually an integer in the range of 0 to 8, selected such that α + β + 1 = γ; at least one ● is a sulfur-containing covalent linker ◊ that is stable under the deprotection condition; optionally the sulfur-containing covalent linker comprises a sulfur-containing cleavable group.

In an embodiment, the disclosure provides a process for preparing a multi-conjugate of Structure 6d comprising deprotecting a compound of Structure 6a. to form a compound of Structure 6b; and reacting the compound of Structure 6b with a compound of Structure 6c under conditions selected to form a compound of Structure 6d, as follows:

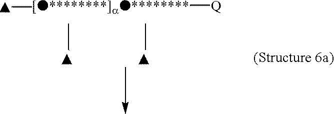

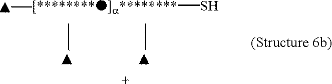

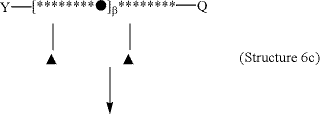

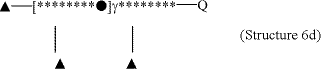

Wherein each of the subunits ******** is independently a bioactive moiety; each ● is a covalent linker; at least one covalent linker ● is a sulfur-containing covalent linker ◇, optionally the sulfur-containing covalent linker ◇ comprises a sulfur-containing cleavable group; each ▲ is independently a group that is absent or comprises a functional moiety joined to a subunit and, optionally, a spacer group joining the functional moiety to the subunit; Q is a group that comprises a sulfur-containing end group, and optionally a spacer group joining Q to the subunit; Y is a reactive group selected to react with the -R-SH group of Structure 6b to form one of the covalent linkers ● of Structure 6d; γ is an integer in the range of 1 to 9; α and β are each individually an integer in the range of 0 to 8, selected such that α + β + 1 = y.

In an embodiment, the disclosure provides a process for preparing a multi-conjugate of Structure 6f comprising deprotecting a compound of Structure 6a to form a compound of Structure 6b; and reacting the compound of Structure 6b with a compound of Structure 6e under conditions selected to form a compound of Structure 6f, as follows:

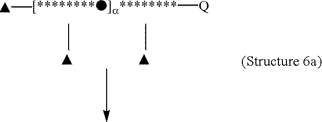

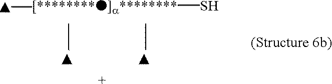

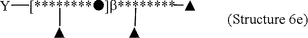

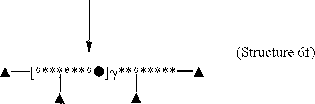

Wherein each of the subunits ******** is independently a bioactive moiety; each ● is a covalent linker; at least one covalent linker ● is a sulfur-containing covalent linker ◇, optionally the sulfur-containing covalent linker ◇ comprises a sulfur-containing cleavable group; each ▲ is independently a group that is absent or comprises a functional moiety and, optionally, a spacer group joining the functional moiety to the subunit; Q is a group that comprises a sulfur-containing end group, and optionally a spacer group joining Q to the subunit; Y is a reactive group selected to react with the -R-SH group of Structure 4b to form one of the covalent linkers ● of Structure 6f; γ is an integer in the range of 1 to 9; α and β are each individually an integer in the range of 0 to 7, selected such that α + β + 1 = γ.

These and other embodiments are described in greater detail below.

### DRAWING

FIG. 1 illustrates reaction Scheme 1 for making a multimeric oligonucleotide. “Lig” indicates a ligand, e.g., as described elsewhere herein with respect to L in Structure 1, such as triantennary GalNAc as described in the Example below. “-S-CL-S-” represents a covalent linker such as an internal DTME linkage as described in the Example below. “Tr” indicates a trityl group and “DTME” indicates a terminal dithiobismaleimidoethane group that reacts with a thiol group to form the -S-CL-S- linker. Solid lines represent single stranded oligonucleotides and dotted lines represent oligonucleotides that are annealed to the multimeric oligonucleotide as described in the Example below.

### DETAILED DESCRIPTION

The disclosures of any patents, patent applications, and publications referred to herein are hereby incorporated by reference in their entireties into this application in order to more fully describe the state of the art known to those skilled herein as of the date of the disclosure described and claimed herein.

### Definitions

As used herein, the term “biological moiety” or “functional moiety” are interchangeable and have ordinary meaning as understood by those skilled in the art. The terms refers to chemical entities that are biologically active or inert when delivered into a cell or organism.

A biological moiety that can produce a biological effect, affinity, or activity within the cell or organism to which it is delivered is referred to as a “bioactive moiety.” In some embodiments the biological effect, affinity, or activity is detectable or measurable. In other instances, a bioactive moiety may be selected to augment or enhance the biological effect, affinity, or activity of another biological moiety with which it is delivered. In still other instances, a bioactive moiety may be selected for use in a method for synthesizing a synthetic intermediate or multi-conjugate (as described below).

Examples of bioactive moieties include but are not limited to nucleic acids, amino acids, peptides, proteins, lipids, carbohydrates, carboxylic acids, vitamins, steroids, lignins, small molecules, organometallic compounds, or derivatives of any of the foregoing.

In some aspects of the disclosure, a “non-nucleic acid biological moiety” refers to any biological moiety other than a nucleic acid. Non-nucleic acid biological moieties include but are not limited to amino acids, peptides, proteins, lipids, carbohydrates, carboxylic acids, vitamins, steroids, lignins, small molecules (e.g., a small molecule therapeutic or drug molecule), organometallic compounds, or derivatives of any of the foregoing. A non-nucleic acid biological moiety that can produce a biological effect or activity within the cell or organism to which it is delivered is referred to as a “non-nucleic acid bioactive moiety.”

“Alkyl” refers to a straight or branched, saturated, aliphatic radical. The number of carbon atoms present in the alkyl group may be specified by number (e.g., C₃ alkyl contains three carbon atoms). The size range of an alkyl group can be specified by indicating a range of the numbers of carbon atoms (e.g., C₁-C₃ alkyl for a one to three carbon atom containing alkyl group). For example, C₁-C₆ alkyl includes, but is not limited to, methyl, ethyl, propyl, isopropyl, butyl, isobutyl, sec-butyl, tert-butyl, pentyl, isopentyl, hexyl, etc. Non-limiting examples of alkyl groups include methyl, ethyl, propyl, butyl, pentyl, 1-methylbutyl (i.e., 2-pentyl), 1-ethylpropyl (i.e., 3-pentyl), 3-methylpentyl, and the like. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6 carbons. The alkyl group is typically monovalent, but can be divalent, such as when the alkyl group links two moieties together, and it is understood that “alkyl” includes alkylene when two functionalities are appended.

“Alkyl ether” refers to a straight or branched chain saturated hydrocarbon containing 1-12 carbon atoms and 1-12 oxygen atoms in the chain. Examples of alkyl ethers include those represented by -((alkyl)—O—)— or —((CH₂)ₙ—O—)ₘ— where n is an integer in the range of 1 to 6 and m is an integer in the range of 1 to 1 2. A polyethylene glycol (PEG) group or linker is an example of an alkyl ether that may be represented by —((CH₂)₂—O—)ₘ—. An “alkoxy” is an example of an alkyl ether that contains a single oxygen atom attached to an end of the alkyl group e.g., —O—(alkyl). Examples of alkoxy groups include without limitation, methoxy, ethoxy, propoxy, butoxy, t-butoxy, or pentoxy groups.

“Aryl” refers to a monocyclic or fused bicyclic, tricyclic or greater, aromatic ring assembly containing 6 to 16 ring carbon atoms. Examples of aryl groups include, but are not limited to, phenyl, naphthyl, phenanthrenyl, naphthacenyl, fluorenyl, pyrenyl, and the like. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C2-C3-alkylene; all of which are optionally further substituted, for instance as hereinbefore defined; or 1- or 2-naphthyl; or 1- or 2-phenanthrenyl.

“Heteroaryl” refers to a monocyclic or fused bicyclic or tricyclic aromatic ring assembly containing 5 to 16 ring atoms, where from 1 to 4 of the ring atoms are each a heteroatom independently selected from N, O and S. Non-limiting examples of heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

“Heterocyclyl” refers to a ring system having from 3 ring members to about 20 ring members and from 1 to about 5 heteroatoms independently selected from N, O and S. For example, heterocyclyl includes, but is not limited to, tetrahydrofuranyl, tetrahydrothiophenyl, morpholino, pyrrolidinyl, pyrrolinyl, imidazolidinyl, imidazolinyl, pyrazolidinyl, pyrazolinyl, piperazinyl, piperidinyl, indolinyl, quinuclidinyl and 1,4-dioxa-8-aza-spiro[4.5]dec-8-yl.

The term “detectable label” as used herein has its ordinary meaning as understood by those skilled in the art. It refers to a chemical group that is attachable to a multi-conjugate and detectable by an imaging technique, such as fluorescence spectroscopy. For example, the detectable label may be a dye that comprises a fluorophore, which, after absorption of energy, emits radiation at a defined wavelength. Many suitable fluorescent labels or dyes are known. For example, Welch et al. (Chem. Eur. J. 5(3):951-960, 1999) discloses dansyl-functionalised fluorescent moieties and Zhu et al. (Cytometry 28:206-211, 1997) describes the use of the fluorescent labels Cy3 and Cy5. Other labels are described in Prober et al. (Science 238:336-341, 1987); Connell et al. (BioTechniques 5(4):342-384, 1987), Ansorge et al. (Nucl. Acids Res. 15(11):4593-4602, 1987) and Smith et al. (Nature 321 :674, 1986). Examples of commercially available fluorescent labels include, but are not limited to, fluorescein, rhodamine (such as TMR, texas red or Rox), alexa, bodipy, acridine, coumarin, pyrene, benzanthracene and cyanine (such as Cy2 or Cy4). Other forms of detectable labels include microparticles, including quantum dots (Empodocles, et al., Nature 399: 126-130, 1999), gold nanoparticles (Reichert et al., Anal. Chem. 72:6025-6029, 2000), microbeads (Lacoste et al., Proc. Natl. Acad. Sci USA 97(17):9461 -9466, 2000), and tags detectable by mass spectrometry. The detectable label may be a multi-component label that is dependent on an interaction with another compound for detection, such as the biotin-streptavidin system.

### Multimeric Oligonucleotides With a Protected Sulfur-Containing End Group

The present disclosure provides a multimeric oligonucleotide comprising a plurality of subunits ******** and a protected sulfur-containing end group. Each of the subunit ******** is independently a single or a double stranded oligonucleotide and is joined to another subunit by a covalent linker ●, and at least one of the covalent linker ● is a sulfur-containing covalent linker ◇. In an embodiment, the sulfer-containing end group comprises a protected thiol group. In some embodiments, the protected sulfur-containing end group comprises a protecting group PG selected from optionally substituted alkyl, optionally substituted alkoxyalkyl, optionally substituted trialkylsilyl, optionally substituted arylalkylsilyl, optionally substituted aryl, optionally substituted benzyl, optionally substituted acyl and optionally substituted benzoyl. In some embodiments, the protected sulfur-containing end group comprises a protecting group PG selected from trityl, methoxytrityl, dimethoxytrityl, methylmethoxy, triisopropylsilyl, dinitrophenyl, nitrophenyl, acetyl, and benzoyl. In some embodiments, the protected thiol is trityl thiol. In some embodiments, the protected sulfur-containing end group does not comprise a thiopropionate group or a disulfide group. The protected sulfur-containing end group is deprotectable under a deprotection condition known to a person of ordinary skill in the art. Each sulfur-containing covalent linker ◇ is stable under the deprotection condition.

In some embodiments, at least one sulfur-containing covalent linker ◇ comprises a cleavable group that is cleavable under an intracellular cleavage condition. Examples of the cleavable group include, but are not limited to, disulfide and thiopropionate. The cleavage condition is known to a person of ordinary skill in the art, and is not the same as the deprotection condition.

In some embodiments, the multimeric oligonucleotide disclosed herein comprises the following structure:

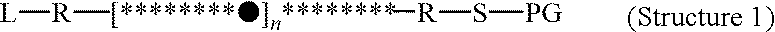

wherein L is a bioacitve moiety that may be present or absent and has biological activity or affinity; each R is individually a spacer group that may be present or absent; each ******** is independently a single or double stranded oligonucleotide subunit; each ● is a covalent linker joining adjacent oligonucleotide subunits; n is an integer in the range of 1 to 9; S-PG is a protected sulfur-containing end group that is deprotectable under a deprotection condition, optionally, S-PG is a protected thiol group; and at least one ● is a sulfur-containing covalent linker ◇ that is stable under the deprotection condition. In some embodiments, the protected sulfur-containing end group does not comprise a thiopropionate group or a disulfide group.

In some embodiments, n is an integer in the range of 2 to 6.

In some embodiments, at least two subunits ******** are substantially different. In some embodiments, the multimeric oligonucleotide comprises two, three, four, five, or six subunits ******** In some embodiments, each nucleic acid strand within a subunit ******** is 5-30, 15-30, 17-27, 19-26, or 20-25 nucleotides in length.

In some embodiments, one or more subunits ******** are a double-stranded RNA. In some embodiments, one or more subunits ******** are a double-stranded RNA. In some embodiments, one or more subunits ******** are a single-stranded RNA. In some embodiments, the subunits ******** comprises a combination of single-stranded and double-stranded oligonucleotides.

In some embodiments, each subunit ******** is an RNA, a DNA, or an artificial or non-natural nucleic acid analog thereof. In some embodiments, each subunit ******** is an siRNA, an saRNA, or a miRNA. In some embodiments, each subunit ******** is a double-stranded siRNA.

In some embodiments, at least one of the covalent linkers ● is a cleavable covalent linker CL, the cleavable covalent linker CL being different from the sulfur-containing covalent linker ◇. In some embodiments, the cleavable covalent linker CL comprises an acid cleavable bond, a reductant cleavable bond, a bio-cleavable bond, or an enzyme cleavable bond. In some embodiments, the cleavable covalent linker CL is cleavable under intracellular conditions.

In some embodiments, at least one of the spacer groups R that is present in the multimeric oligonucleotide of Structure 1 comprises alkyl, alkyl ether, ester, aryl, heteroaryl, heterocyclyl, alkyl-aryl, alkyl-heteroaryl, or alkyl-heterocyclyl. In some embodiments, every spacer group R that is present in the multimeric oligonucleotide of Structure 1 comprises alkyl, alkyl ether, ester, aryl, heteroaryl, heterocyclyl, alkyl-aryl, alkyl-heteroaryl, or alkyl-heterocyclyl. In some embodiments, at least one of the spacer groups R that is present in the multimeric oligonucleotide of Structure 1 comprises C₁-₁₀ alkyl, C₁-₁₀ alkyl ether, C₁₋₁₀ alkyl ester, 6-10 membered aryl, 5-10 membered heteroaryl, 5-10 membered heterocyclyl, (C₁-₁₀ alkyl)-(6-10 membered aryl), (C₁-₁₀ alkyl)-(5-10 membered heteroaryl), or (C₁-₁₀ alkyl)-(5-1 0 membered heterocyclyl). In some embodiments, every spacer group R that is present in the multimeric oligonucleotide comprises C₁₋₁₀ alkyl, C₁-₁₀ alkyl ether, C₁-₁₀ alkyl ester, 6-10 membered aryl, 5-10 membered heteroaryl, 5-10 membered heterocyclyl, (C₁₋₁₀-alkyl)-(6-10 membered aryl), (C₁-₁₀ alkyl)-(5-10 membered heteroaryl), or (C₁₋₁₀alkyl)-(5-10 membered heterocyclyl).

In some embodiments, at least one of the spacer groups R that is present in the multimeric oligonucleotide of Structure 1 comprises C₂-C₁₀ alkyl, C₂-C₁₀ alkyl ether, C₂-C₁₀ alkyl ester, or C₆-C₁₀ aryl. In some embodiments, every spacer group R that is present in the multimeric oligonucleotide comprises C₂-C₁₀ alkyl, C₂-C₁₀ alkyl ether, C₂-C₁₀ alkyl ester, or C₆-C₁₀ aryl.

In some embodiments, at least one of the spacer groups R that is present in the multimeric oligonucleotide of Structure 1 comprises C₂, C₃, C₄, C₅, or C₆ alkyl. In some embodiments, every spacer group R that is present in the multimeric oligonucleotide comprises C₂, C₃, C₄, C₅, or C₆ alkyl.

In some embodiments, at least one of the spacer groups R that is present in the multimeric oligonucleotide of Structure 1 comprises C₆ alkyl. In some embodiments, every spacer group R that is present in the multimeric oligonucleotide comprises C₆ alkyl.

In some embodiments, at least one of the spacer groups R that is present in the multimeric oligonucleotide of Structure 1 comprises 1,4-phenylene. In some embodiments, every spacer group R that is present in the multimeric oligonucleotide comprises 1,4-phenylene.

In some embodiments, the sulfur-containing covalent linker 0 comprises a linkage represented by -R¹-R²-R¹-, wherein each R¹ is individually absent or a spacer group, and R² is a thiopropionate or disulfide group. In some embodiments, the protected thiol group does not comprise a thiopropionate group or a disulfide group.

In some embodiments, at least one of the spacer groups R¹ that is present in the linkage comprises alkyl, alkyl ether, ester, aryl, heteroaryl, heterocyclyl, alkyl-aryl, alkyl-heteroaryl, or alkyl-heterocyclyl. In some embodiments, every spacer group R¹ that is present in the linkage comprises alkyl, alkyl ether, ester, aryl, heteroaryl, heterocyclyl, alkyl-aryl, alkyl-heteroaryl, or alkyl-heterocyclyl.

In some embodiments, at least one of the spacer groups R¹ that is present in the linkage comprises C1-₁₀ alkyl, C1-₁₀ alkyl ether, C1-₁₀ alkyl ester, 6-1 0 membered aryl, 5-10 membered heteroaryl, 5-10 membered heterocyclyl, (C₁-₁₀ alkyl)-(6-10 membered aryl), (C₁-₁₀ alkyl)-(5-10 membered heteroaryl), or (C₁-₁₀ alkyl)-(5-10 membered heterocyclyl). In some embodiments, every spacer group _{R1} that is present in the linkage comprises C₁-₁₀ alkyl, C1-₁₀ alkyl ether, C₁-₁₀alkyl ester, 6-10 membered aryl, 5-10 membered heteroaryl, 5-10 membered heterocyclyl, (C₁-₁₀alkyl)-(6-10 membered aryl), (C₁-₁₀alkyl)-(5-10 membered heteroaryl), or (C₁₋₁₀alkyl)-(5-10 membered heterocyclyl).

In some embodiments, at least one of the spacer groups R¹ that is present in the linkage comprises C₂-C₁₀ alkyl, C₂-C₁₀ alkyl ether, C₂-C₁₀ alkyl ester, or C₆-C₁₀ aryl; optionally wherein every spacer group R1 that is present in the multimeric oligonucleotide comprises C₂-C₁₀ alkyl, C₂-C₁₀ alkyl ether, C₂-C₁₀ alkyl ester, or C₆-C_{1O} aryl.

In some embodiments, at least one of the spacer groups R¹ that is present in the linkage comprises C₂. C₃, C₄, C5, or C₆ alkyl. In some embodiments, every spacer group R¹ that is present in the linkage comprises C₂, C₃, C₄, C₅, or C₆ alkyl.

In some embodiments, at least one of the spacer groups R¹ that is present in the linkage comprises C₆ alkyl. In some embodiments, every spacer group R¹ that is present in the linkage comprises C₆ alkyl.

In some embodiments, at least one of the spacer groups R¹ that is present in the linkage comprises 1,4-phenylene. In some embodiments, every spacer group R¹ that is present in the linkage comprises 1,4-phenylene.

In some embodiments, at least one of the spacer groups R¹ that is present in the linkage comprises a phosphate linking group, a phosphorothioate linking group, a phosphonate linking group, or a dithiophosphate linking group. In some embodiments, every spacer group R¹ that is present in the linkage comprises a phospliate linking group, a phosphorothioate linking group, a phosphonate linking group, or a dithiophosphate linking group.

In some embodiments, at least one of the spacer groups R¹ that is present in the linkage comprises a linking group represented by

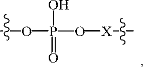

wherein each X independently comprises alkyl, alkyl ether, ester, aryl, heteroaryl, heterocyclyl, alkyl-aryl, alkyl-heteroaryl, or alkyl-heterocyclyl. In some embodiments, every spacer group R¹ that is present in the linkage comprises a linking group represented by

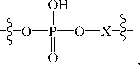

wherein each X independently comprises alkyl, alkyl ether, ester, aryl, heteroaryl, heterocyclyl, alkyl-aryl, alkyl-heteroaryl, or alkyl-heterocyclyl.

In some embodiments, at least one of the spacer groups R1 that is present in the linkage comprises a pyrrolidine-2,5-dione. In some embodiments, every spacer group R¹ that is present in the linkage comprises a pyrrolidine-2,-dione.

In some embodiments, the linkage represented by -R¹-R²-R¹- can also be represented by:

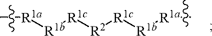

wherein each R^{1a} is independently absent,

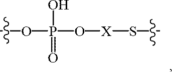

or

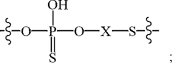

each R^{1b} is independently absent,

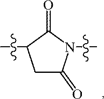

or

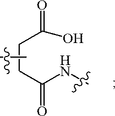

each R^{1c} is X; and R² is a thiopropionate or disulfide group. Each X independently comprises alkyl, alkyl ether, ester, aryl, heteroaryl, heterocyclyl, alkyl-aryl, alkyl-heteroaryl, or alkyl-heterocyclyl.

The linking group

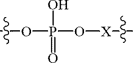

also includes

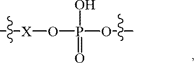

and

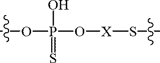

also includes

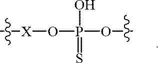

X in the linking group would be the moiety that is connected to R^{1b}.

In some embodiments, each X independently comprises C1-₁₀ alkyl, C₁-₁₀ alkyl ether, C₁-₁₀alkyl ester, 6-10 membered aryl, 5-10 membered heteroaryl, 5-10 membered heterocyclyl, (C₁-₁₀ alkyl)-(6-1 0 membered aryl), (C1-10 alkyl)-(5-10 membered heteroaryl), or (C1,10alkyl)-(5-10 membered heterocyclyl). In some embodiments, each X independently comprises C₂-C₁₀ alkyl, C₂-C₁₀ alkyl ether, C₂-C₁₀ alkyl ester, or C₆-C_{10aryl} In some embodiments, each X independently comprises C₂, C₃, C₄, C₅, or C₆ alkyl. In some embodiments, each X comprises C₆ alkyl. In some embodiments, each X comprises 1,4-phenylene.

In some embodiments, the linkage represented by -R¹-R²-R¹ comprises

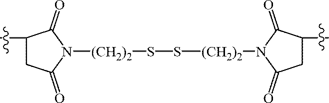

or a ring-opened derivative thereof, such as

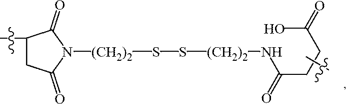

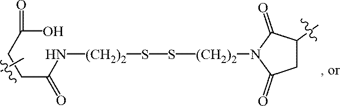

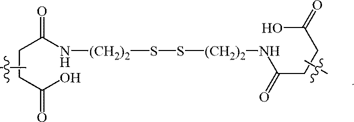

In some embodiments, the linkage represented by -R¹-R^{2_}R¹- is:

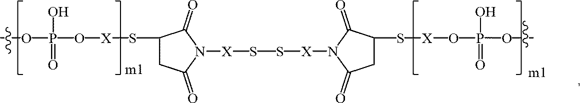

or a ring-opened derivative thereof, wherein each X is independently as defined above, m1 are each individually an integer in the range of 1 to 10.

In some embodiments, the linkage represented by -R¹-R²-R¹- is:

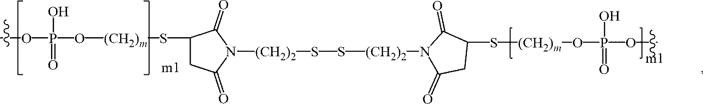

or a ring-opened derivative thereof; wherein m and m1 are each individually an integer in the range of 1 to 10.

In some embodiments, L comprises a targeting ligand. In some embodiments, the targeting ligand comprises an aptamer, N-Acetylgalactosamine (GalNAc), folate, lipid, cholesterol, or transferrin. In some embodimetns, L comprises an endosomal escape moiety. In some embodiments, the endosomal escape moiety is a membrane disrupting, altering, or destabilizing peptide, lipid, polymer, or small molecule.

In some embodiments, L comprises a detectable label. In some embodiments, the detectable label selected from fluorescein, a rhodamine (such as TMR, texas red or Rox), alexa, bodipy, acridine, coumarin, pyrene, benzanthracene and a cyanine (such as Cy2 or Cy4). For example, in an embodiment, the detectable labels are Cy2 and Cy4.into a light drug, a rhodamine (such as TMR, texas red or Rox), alexa, bodipy, acridine, coumarin, pyrene, benzanthracene and a cyanine (such as Cy2 or Cy4). For example, in an embodiment, the detectable labels are Cy2 and Cy4.

### Method of Making Multimeric Oligonucleotides

The multimeric oligonucleotides described herein may be made in various ways. The disclosure provides a process for preparing a multimeric oligonucleotide as described herein. The process includes deprotecting a compound of Structure 1a to form a compound of Structure 1b; and reacting the compound of Structure 1b with a compound of Structure 1c under conditions selected to form a compound of Structure 1d, as follows:

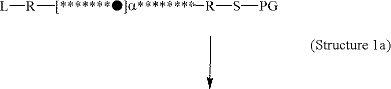

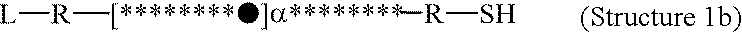

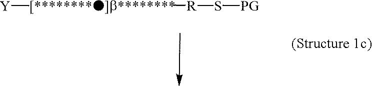

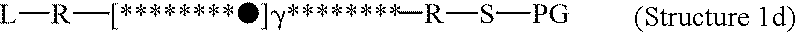

wherein L is a bioactive moiety that may be present or absent; each R is individually a spacer group that may be present or absent; each ******** is independently a single or double stranded oligonucleotide; each ● is a covalent linker joining adjacent oligonucleotide subunits; S-PG is a protected sulfur-containing end group, optionally a protected thiol group, that is deprotectable under a deprotection condition; Y is a reactive group selected to react with the —R—SH group of Structure 1b to form one of the covalent linkers » of Structure 1d; y is an integer in the range of 1 to 9; cr, and are each individually an integer in the range of 0 to 8, selected such that a + P + 1 = y; and at least one o is a sulfur-containing covalent linker 0 that is stable under the deprotection condition.

The disclosure provides a process for preparing another multimeric oligonucleotide as described herein. The process includes deprotecting a compound of Structure 1a to form a compound of Structure 1b; and reacting the compound of Structure lb with a compound of Structure 1e under conditions selected to form a compound of Structure 1f, as follows:

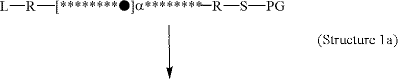

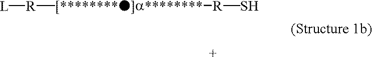

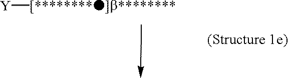

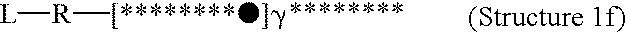

wherein L is a moiety that may be present or absent and has biological activity or affinity; each R is individually a spacer group that may be present or absent; each ******** is independently a single or double stranded oligonucleotide. Each » is a covalent linker joining adjacent oligonucleotide subunits; S-PG is a protected sulfur-containing end group, optionally a protected thiol group, that is deprotectable under a deprotection condition; Y is a reactive group selected to react with the —R—SH group of Structure 2b to form one of the covalent linkers ● of Structure If; y is an integer in the range of 1 to 9; a and β are each individually an integer in the range of 0 to 8, selected such that a + 0 + 1 = y; at least one ● is a sulfur-containing covalent linker 0 that is stable under the deprotection condition.

In some embodiments, the spacer groups R that is present in the multimeric oligonucleotide of Structure 1 comprises alkyl, alkyl ether, ester, aryl, heteroaryl, heterocyclyl, alkyl-aryl, alkyl-heteroaryl, or alkyl-heterocyclyl. In some embodiments, every spacer group R that is present in the multimeric oligonucleotide of Structure 1 comprises alkyl, alkyl ether, ester, aryl, heteroaryl, heterocyclyl, alkyl-aryl, alkyl-heteroaryl, or alkyl-heterocyclyl. In some embodiments, at least one of the spacer groups R that is present in the multimeric oligonucleotide of Structure 1 comprises C₁₋₁₀alkyl, C₁₋₁₀alkyl ether, C₁₋₁₀alkyl ester, 6-10 membered aryl, 5-10 membered heteroaryl, 5-10 membered heterocyclyl, (C₁ -₁₀ alₖyl)-( 6-10 membered aryl), (C_{1-10alky}(5-₁0 membered heteroaryl), or (C_{1-10 alky}10 membered heterocyclyl). In some embodiments, every spacer group R that is present in the multimeric oligonucleotide comprises C₁₋₁₀alkyl, C₁₋₁₀alkyl ether, C₁₋₁₀alkyl ester, 6-10membered aryl, 5-10 membered heteroaryl, 5-10 membered heterocyclyl, (C_{1-10alkyl}-(6-10 membered aryl), (C₁₋₁₀ alkyl)-(5-10 membered heteroaryl), or (C₁₋₁₀ alkyl(5-10membered heterocyclyl).

In some embodiments, at least one of the spacer groups R that is present in the multimeric oligonucleotide of Structure 1 comprises C₂-C₁₀ alkyl, C₂-C₁₀ alkyl ether, C₂-C₁₀ alkyl ester, or C₆-C₁₀ aryl. In some embodiments, every spacer group R that is present in the multimeric oligonucleotide comprises C₂-C₁₀ alkyl, C₂-C₁₀ alkyl ether, C₂-C₁₀ alkyl ester, or C₆-C1₀ aryl.

In some embodiments, at least one of the spacer groups R that is present in the multimeric oligonucleotide of Structure 1 comprises C_{2,} C₃, C₄, C₅, or C₆ alkyl. In some embodiments, every spacer group R that is present in the multimeric oligonucleotide comprises C₂, C₃, C₄, C₅, or C₆ alkyl.

In some embodiments, at least one of the spacer groups R that is present in the multimeric oligonucleotide of Structure 1 comprises C₆ alkyl. In some embodiments, every spacer group R that is present in the multimeric oligonucleotide comprises C₆ alkyl.

In some embodiments, at least one of the spacer groups R that is present in the multimeric oligonucleotide of Structure 1 comprises 1,4-phenylene. In some embodiments, every spacer group R that is present in the multimeric oligonucleotide comprises 1,4-phenylene.

In some embodiments, the sulfur-containing covalent linker 0 comprises a linkage represented by -R¹-R²-R¹-, wherein each R¹ is individually absent or a spacer group, and R² is a thiopropionate or disulfide group. In some embodiments, the protected thiol group does not comprise a thiopropionate group or a disulfide group.

In some embodiments, at least one of the spacer groups R¹ that is present in the linkage comprises alkyl, alkyl ether, ester, aryl, heteroaryl, heterocyclyl, alkyl-aryl, alkyl-heteroaryl, or alkyl-heterocyclyl. In some embodiments, every spacer group R¹ that is present in the linkage comprises alkyl, alkyl ether, ester, aryl, heteroaryl, heterocyclyl, alkyl-aryl, alkyl-heteroaryl, or alkyl-heterocyclyl.

In some embodiments, at least one of the spacer groups R¹ that is present in the linkage comprises C₁₋₁₀ alkyl, C₁-₁₀ alkyl ether, C₁₋₁₀ alkyl ester, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁-₁₀alkyl)-(5-10 membered heterocyclyl). In some embodiments, every spacer group R₁ that is present in the linkage comprises C₁₋₁₀ alkyl, C₁₋₁₀ alkyl ether, C₁₋₁₀ alkyl ester, 6-10 membered aryl, 5-10 membered heteroaryl, 5-10 membered heterocyclyl, (C₁₋₁₀alkyl)-(6-10 membered aryl), (C₁-₁₀alkyl)-(5-10 membered heteroaryl), or (C₁₋₁₀alkyl)-(5-10 membered heterocyclyl).

In some embodiments, at least one of the spacer groups R¹ that is present in the linkage comprises C₂-C₁₀ alkyl, C₂-C₁₀ alkyl ether, C₂-C₁₀ alkyl ester, or C₆-C₁₀ aryl; optionally wherein every spacer group Rthat is present in the multimeric oligonucleotide comprises C₂-C₁₀ alkyl, C₂-C₁₀ alkyl ether, C₂-C₁₀ alkyl ester, or C₆-C₁₀ aryl.

In some embodiments, at least one of the spacer groups R¹ that is present in the linkage comprises C₂, C₃, C₄, C₅, or C₆ alkyl. In some embodiments, every spacer group R¹ that is present in the linkage comprises C₂, C₃, C₄, C₅, or C₆ alkyl.

In some embodiments, at least one of the spacer groups R¹ that is present in the linkage comprises C₆ alkyl. In some embodiments, every spacer group R¹ that is present in the linkage comprises C₆ alkyl.

In some embodiments, at least one of the spacer groups R¹ that is present in the linkage comprises 1,4-phenylene. In some embodiments, every spacer group R¹ that is present in the linkage comprises 1,4-phenylene.

In some embodiments, at least one of the spacer groups R¹ that is present in the linkage comprises a phosphate linking group, a phosphorothioate linking group, a phosphonate linking group, or a dithiophosphate linking group. In some embodiments, every spacer group R¹ that is present in the linkage comprises a phosphate linking group, a phosphorothioate linking group, a phosphonate linking group, or a dithiophosphate linking group.

In some embodiments, at least one of the spacer groups R¹ that is present in the linkage comprises a linking group represented by

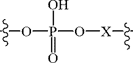

, wherein each X independently comprises alkyl, alkyl ether, ester, aryl, heteroaryl, heterocyclyl, alkyl-aryl, alkyl-heteroaryl, or alkyl-heterocyclyl. In some embodiments, every spacer group R¹ that is present in the linkage comprises a linking group represented by

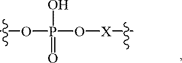

wherein each X independently comprises alkyl, alkyl ether, ester, aryl, heteroaryl, heterocyclyl, alkyl-aryl, alkyl-heteroaryl, or alkyl-heterocyclyl. In some embodiments, R¹ may comprise a linking group represented by

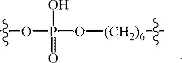

In some embodiments, at least one of the spacer groups R¹ that is present in the linkage comprises a pyrrolidine-2,5-dione. In some embodiments, every spacer group R¹ that is present in the linkage comprises a pyrrolidine-2,5-dione.

In some embodiments, the linkage represented by -R¹-R²-R¹- can also be represented by:

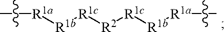

wherein each R^{1a} is independently absent,

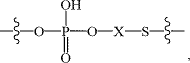

or

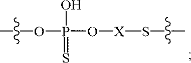

each R^{1b} is independently absent,

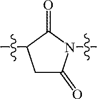

, or

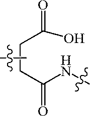

; each R^{1c} is X; and R² is a thiopropionate or disulfide group. Each X independently comprises alkyl, alkyl ether, ester, aryl, heteroaryl, heterocyclyl, alkyl-aryl, alkyl-heteroaryl, or alkyl-heterocyclyl.

In some embodiments, each R^{1a} is independently absent, or is present and is

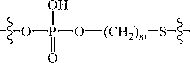

, or

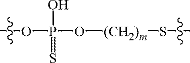

, where m is an integer in the range of 1 to 10;

In some embodiments, each X independently comprises C₁-₁₀ alkyl, C₁-₁₀ alkyl ether, C₁₋₁₀alkyl ester, 6-10 membered aryl, 5-10 membered heteroaryl, 5-10 membered heterocyclyl, (C₁₋₁₀alkyl)-(6-10 membered aryl), (C₁₋₁₀alkyl)-(5-10 membered heteroaryl), or (C₁₋₁₀ alkyl)-(5-10 membered heterocyclyl). In some embodiments, each X independently comprises C₂-C₁₀ alkyl, C₂-C₁₀ alkyl ether, C₂-C₁₀ alkyl ester, or C₆-C₁₀ aryl. In some embodiments, each X independently comprises C₂, C₃, C₄, C₅, or C₆ alkyl. In some embodiments, each X comprises C₆ alkyl. In some embodiments, each X comprises 1,4-phenylene,

In some embodiments, the linkage represented by -R¹-R²-R¹- comprises

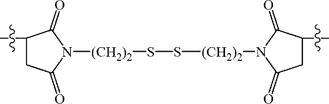

or a ring-opened derivative thereof, such as

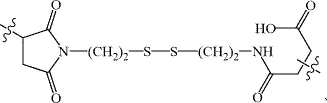

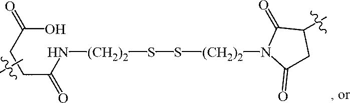

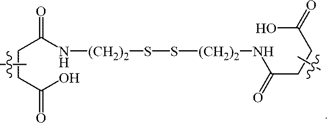

In some embodiments, the linkage represented by -R¹-R²-R¹- is:

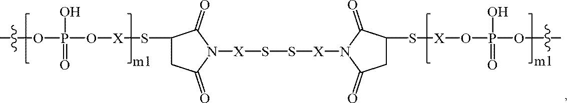

or a ring-opened derivative thereof; wherein each X is independently as defined above, m1 are each individually an integer in the range of 1 to 10.

In some embodiments, the linkage represented by -R¹-R²-R¹- is:

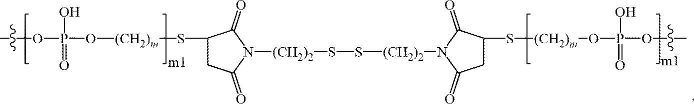

, or a ring-opened derivative thereof; wherein m and ml are each individually an integer in the range of 1 to 10.

In some embodiments, L comprises a targeting ligand. In some embodiments, the targeting ligand comprises an aptamer, N-Acetylgalactosamine (GalNAc), folate, lipid, cholesterol, or transferrin. In some embodiments, L comprises an endosomal escape moiety. In some embodiments, the endosomal escape moiety is a membrane disrupting, altering, or destabilizing peptide, lipid, polymer, or small molecule.

In some embodiments, L comprises a detectable label. In some embodiments, the detectable label selected from fluorescein, a rhodamine (such as TMR, texas red or Rox), alexa, bodipy, acridine, coumarin, pyrene, benzanthracene and a cyanine (such as Cy2 or Cy4). For example, in an embodiment, the detectable labels are Cy2 and Cy4.into a light drug, a rhodamine (such as TMR, texas red or Rox), alexa, bodipy, acridine, coumarin, pyrene, benzanthracene and a cyanine (such as Cy2 or Cy4). For example, in an embodiment, the detectable labels are Cy2 and Cy4.

In some embodiments, Y is a reactive group represented by

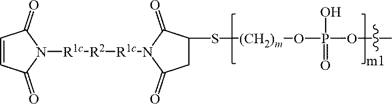

or a ring-opened derivative thereof, wherein each R^{1c} is independently C₁₋₁₀ alkylene or C₁₋₁₀alkyleneoxy; R² is a thiopropionate or disulfide group; m is an integer in the range of 1 to 10; and ml is an integer in the range of 1 to 10.

In some embodiments, Y is a reactive group represented by

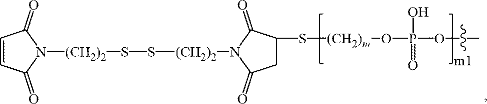

or a ring-opened derivative thereof.

### Multi-Conjugates

The disclosure also provides a multi-conjugate comprising a plurality of subunits ******** joined to one another by one or more covalent linkers ●, wherein the multi-conjugate comprises Structure 4:

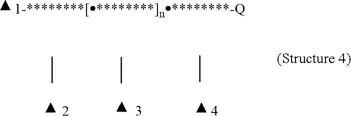

wherein each of the subunits ******** is independently a bioactive moiety; at least one covalent linker ● is a sulfur-containing covalent linker ◊; at least one covalent linker ● is a sulfur-containing covalent linker ◊; each of ▲₁, ▲₂, ▲₃, and▲₄ is a group that is independently absent or comprises a functional moiety joined to a subunit and, optionally, a spacer group joining the functional moiety to the subunit; Q is a group that comprises a sulfur-containing end group, and optionally a spacer group joining Q to the subunit; and n is an integer greater than or equal to zero. In some emboidments, n is an integer in the range of 0 to 10. In some embodiments, n is an integer in the range of 1 to 4. In some embodiments, n is 1, 2, 3, or 4.

In some embodiments, ▲ ₂, ▲₃, and ▲₄ are absent.

In some embodiments, at least one of the subunits ******** present in Structure 4 is not an oligonucleotide. In some embodiments, at least one of the subunits ******** present in Structure 4 comprises an oligopeptide or a protein.

In some embodiments, at least one functional moiety is present. In some embodiments, at least one functional moiety that is present is a targeting ligand. In some embodiments, the at least one functional moiety that is present is a detectable label; optionally, the detectable label is a dye.

In some embodiments, the sulfur-containing end group Q comprises a protected thiol group that is deprotectable under a deprotection condition; and the sulfur-containing covalent linker ◊ is stable under the deprotection condition. In some embodiments, the sulfur-containing covalent linker ◊ comprises a sulfur-containing cleavable group, including but not limited to C₂-C₁₀ alkyldiothio, thioether, thiopropionate, or disulfide. In an embodiment, the sulfur-containing covalent linker ◊ comprises a sulfur-containing cleavable group that is cleavable under a cleavage condition that is not the deprotection condition.

In some embodiments, the sulfur-containing end group Q comprises a protected thiol group.

In some embodiments, at least one covalent linker ● comprises Structure 5:

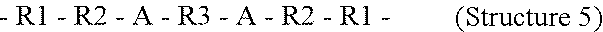

wherein each R1 is independently a group comprising phosphodiester, thiophosphodiester, sulfate, amide, triazole, heteroaryl, ester, ether, thioether, disulfide, thiopropionate, acetal, glycol, or is absent; each R2 is independently a spacer group, or is absent; each A is independently the reaction product of a nucleophile and an electrophile; and R3 is a group comprising a C2-C10 alkyl, C2-C10 alkoxy, C1-C10 aryl, amide, C2-C10 alkyldithio, ether, thioether, ester, oligonucleotide, oligopeptide, thiopropionate, or disulfide. In some embodiments, each A is the same. In some embodiments, R3 of Structure 5 comprises a sulfur-containing group. In some embodiments, R3 comprises a sulfur-containing cleavable group including C₂-C₁₀ alkyldithio, thioether, thiopropionate, or disulfide.

In various embodiments, a multi-conjugate as described herein comprises one or more targeting ligands. The targeting ligand(s) may be attached to one or more of the subunits by a suitable linker. Examples of ligands that can be targeting ligands include antibody, antibody fragment, double chain Ab fragment, single chain Ab fragment; other proteins, for example, a glycoprotein (e.g., transferrin) or a growth factor; peptide (e.g., the RGD ligand or gastrin-releasing peptides); nucleic acid (e.g., an aptamer), endosomal escape moiety (e.g., peptide or nucleic acid), peptide derivative (e.g., DUPA); a natural or synthetic carbohydrate, for example, a monosaccharide (e.g., galactose, mannose, N-Acetylgalactosamine [“GalNAc”]), polysaccharide, or a cluster such as lectin binding oligo saccharide, diantennary GaiNAc, or triantennary GalNAc; a lipid, for example, a sterol (e.g., cholesterol), phospholipid (e.g., phospholipid ether, phosphatidylcholine, lecithin); a vitamin compound (e.g., tocopherol or folate); immunostimulant (e.g., a CpG oligonucleotide); an amino acid; an element (e.g., gold); or a synthetic small molecule (e.g., anisamide or polyethylene glycol). For example, in various embodiments the targeting ligand is an aptamer, N-Acetylgalactosamine (GalNAc), folate, lipid, cholesterol, or transferrin.

### Method of Making Multi-Conjugates

This disclosure provides a method for making a multi-conjugate. The method includes deprotecting a compound of Structure 6a to form a compound of Structure 6b; and reacting the compound of Structure 6b with a compound of Structure 6c under conditions selected to form a compound of Structure 6d, as follows:

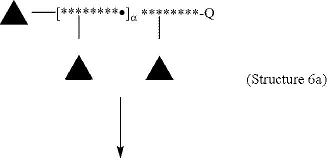

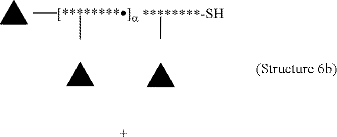

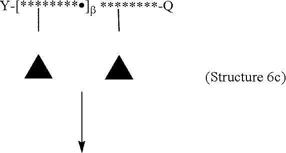

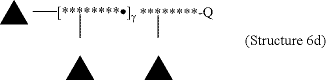

wherein each of the subunits ******** is independently a bioactive moiety; each ● is a covalent linker; at least one covalent linker ● is a sulfur-containing covalent linker ◊; each ▲ is independently a group that is absent or comprises a functional moiety joined to a subunit and, optionally, a spacer group joining the functional moiety to the subunit; Q is a group that comprises a protected sulfur-containing end group (optionally, a protected thiol) that is deprotectable under a deprotection condition and optionally a spacer group joining Q to the subunit; Y is a reactive group selected to react with the —R—SH group of Structure 6b to form one of the covalent linkers ● of Structure 6d; γ is an integer in the range of 1 to 9; α and β are each individually an integer in the range of 0 to 8, selected such that α + β + 1 = γ; and at least one ● is a sulfur-containing covalent linker ◊ that is stable under the deprotection condition.

### Methods of Treatment

In various aspects, the disclosure provides methods for using multimeric oligonucleotides made by the process disclosed herein, for example for medical treatments, research, or for producing new or altered phenotypes in animals and plants. In some aspects, the disclosure also provides methods for using the multi-conjugates made by the process disclosed herein, for example for medical treatments, research, or for producing new or altered phenotypes in animals and plants.

In one aspect, the invention provides a method for treating a subject comprising administering an effective amount of a multimeric oligonucleotides or multi-conjugates according to the disclosure to a subject in need thereof.

In some embodiments, the multimeric oligonucleotides or multi-conjugates made by the processes disclosed herein can be administered in the form of a pharmaceutical composition.

### EXAMPLE

### Example 1. Synthesis of Disulfide-linked Multimeric Oligonucleotides Using Orthogonally Protected Thiols

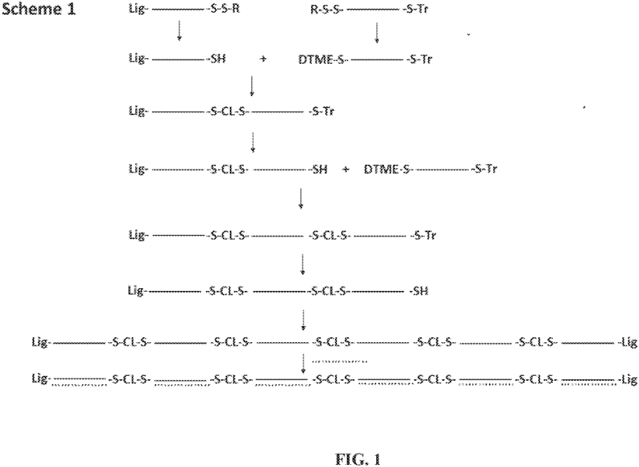

A bis-(triantennary GalNAc) homo-hexamer of TTR siRNA (siTTR) is prepared as outlined in Scheme 1 (FIG. 1), Two monomers of siTTR sense strand are prepared on the synthesizer, one with a terminal amino group, the other with a terminal tritylated thiol. Both have a disulfide group at the other terminus. A triantennary GalNAc group is added to the terminal amino function of the first monomer and then the disulfide groups of both monomers are cleaved by DTT to the corresponding thiols.

The tritylated monomer is converted to a mono-DTME derivative by previously reported methods (see PCT Publication No. WO 2016/205410) and part of this material is reacted with the GalNAc-siTTR-thiol to yield a GalNAc-siTTR single-stranded homodimer with an internal DIME linkage (-S-CL-S-) and a terminal thiol protected by a trityl group.

The trityl group is removed from the homo-dimer by treatment with aqueous silver nitrate and after purification is treated with one molar equivalent of the tritylated mono-DTME derivate to yield a GalNAc-siTTR single-stranded homotrimer with two internal DTME linkages (-S-CL-S-) and a terminal thiol protected by a trityl group.

The trityl group is removed from the homo-trimer by treatment with aqueous silver nitrate and after purification is treated with one half-molar equivalent of DTME to yield the single stranded homo-hexamer. Annealing of six equivalents of TTR antisense siRNA yields the desired bis-(triantennary GalNAc) homo-hexamer of siTTR containing 5 disulfide linkages.

What is claimed is:

1. A multimeric oligonucleotide comprising a plurality of subunits ******** and a protected sulfur-containing end group, wherein:
each of the subunits ******** is independently a single or double stranded oligonucleotide and is joined to another subunit by a covalent linker ●;
at least one of the covalent linker ● is a sulfur-containing covalent linker 0, optionally the sulfur-containing covalent linker comprises a sulfur-containing cleavable group;
the sulfur-containing end group is deprotectable under a deprotection condition; and
each sulfur-containing covalent linker 0 is stable under the deprotection condition.

2. The multimeric oligonucleotide of claim 1, wherein at least one sulfur-containing covalent linker 0 comprises a cleavable group, optionally a sulfur-containing cleavable group, that is cleavable under a cleavage condition that is not the deprotection condition.

3. The multimeric oligonucleotide of claim 1, wherein the protected sulfur-containing end group comprises a protected thiol group.

4. The multimeric oligonucleotide of claim 1 comprising the following Structure 1: 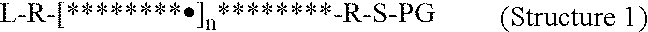
wherein:
L is a bioactive moiety that may be present or absent;
each R is individually a spacer group that may be present or absent;
each ******** is independently a single or double stranded oligonucleotide subunit;
each ● is a covalent linker joining adjacent oligonucleotide subunits;
n is an integer in the range of 1 to 9;
S-PG is a protected sulfur-containing end group, optionally a protected thiol group, that is deprotectable under a deprotection condition; and
at least one ● is a sulfur-containing covalent linker 0, optionally the sulfur-containing covalent linker comprises a sulfur-containing cleavable group, that is stable under the deprotection condition.

5. The multimeric oligonucleotide of claim 4, wherein at least one of the spacer groups R that is present in the multimeric oligonucleotide comprises alkyl, alkyl ether, ester, aryl, heteroaryl, heterocyclyl, alkyl-aryl, alkyl-heteroaryl, or alkyl-heterocyclyl; optionally wherein every spacer group R that is present in the multimeric oligonucleotide comprises alkyl, alkyl ether, ester, aryl, heteroaryl, heterocyclyl, alkyl-aryl, alkyl-heteroaryl, or alkyl-heterocyclyl.

6. The multimeric oligonucleotide of claim 5, wherein at least one of the spacer groups R that is present in the multimeric oligonucleotide comprises C₁₋₁₀ alkyl, C₁₋₁₀ alkyl ether, C₁₋₁₀ alkyl ester, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl); optionally wherein every spacer group R that is present in the multimeric oligonucleotide comprises C₁₋₁₀ alkyl, C₁₋₁₀ alkyl ether, C₁₋₁₀ alkyl ester, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl).

7-10. (canceled)

11. The multimeric oligonucleotide of claim 1, wherein the sulfur-containing covalent linker 0 comprises a linkage represented by -R¹-R²-R¹-, wherein:
each R¹ is individually absent or a spacer group; and
R² is a thiopropionate or disulfide group.

12. (canceled)

13. The multimeric oligonucleotide of claim 11, wherein at least one of the spacer groups R¹ that is present in the linkage comprises alkyl, alkyl ether, ester, aryl, heteroaryl, heterocyclyl, alkyl-aryl, alkyl-heteroaryl, or alkyl-heterocyclyl; optionally wherein every spacer group R¹ that is present in the linkage comprises alkyl, alkyl ether, ester, aryl, heteroaryl, heterocyclyl, alkyl-aryl, alkyl-heteroaryl, or alkyl-heterocyclyl.

14. The multimeric oligonucleotide of claim 13, wherein at least one of the spacer groups R¹ that is present in the linkage comprises C₁₋₁₀ alkyl, C₁₋₁₀ alkyl ether, C₁₋₁₀ alkyl ester, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl); optionally wherein every spacer group R₁ that is present in the linkage comprises C₁₋₁₀ alkyl, C₁₋₁₀ alkyl ether, C₁₋₁₀ alkyl ester, 6-10 membered aryl, 5-10 membered heteroaryl, 5-10 membered heterocyclyl, (C₁₋₁₀ alkyl)-(6-10 membered aryl), (C₁₋₁₀ alkyl)-(5-10 membered heteroaryl), or (C₁₋₁₀ alkyl)-(5-10 membered heterocyclyl).

15-18. (canceled)

19. The multimeric oligonucleotide of claim 11, wherein at least one of the spacer groups R¹ that is present in the linkage comprises a phosphate linking group, a phosphorothioate linking group, a phosphonate linking group, or a dithiophosphate linking group; optionally wherein every spacer group R¹ that is present in the linkage comprises a phosphate linking group, a phosphorothioate linking group, a phosphonate linking group, or a dithiophosphate linking group.

20-29. (canceled)

30. The multimeric oligonucleotide of claim 1, wherein the protected sulfur-containing end group comprises a protecting group selected from optionally substituted alkyl, optionally substituted alkoxyalkyl, optionally substituted trialkylsilyl, optionally substituted arylalkylsilyl, optionally substituted aryl, optionally substituted benzyl, optionally substituted acyl and optionally substituted benzoyl.

31. The multimeric oligonucleotide of claim 1, wherein the protected sulfur-containing end group comprises a protecting group PG selected from trityl, methoxytrityl, dimethoxytrityl, methylmethoxy, triisopropylsilyl, dinitrophenyl, nitrophenyl, acetyl and benzoyl.

32-99. (canceled)

100. A multi-conjugate comprising a plurality of subunits ******** joined to one another by one or more covalent linkers ●, wherein the multi-conjugate comprises Structure 3: 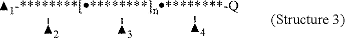
wherein:
each of the subunits ******** is independently a bioactive moiety;
at least one covalent linker • is a sulfur-containing covalent linker 0, optionally the sulfur-containing covalent linker 0 comprises a sulfur-containing cleavable group ;
each of ▲₁, ▲₂, ▲₃, and ▲₄ is a group that is independently absent or comprises a functional moiety joined to a subunit and, optionally, a spacer group joining the functional moiety to the subunit;
Q is a group that comprises a sulfur-containing end group, and optionally a spacer group joining Q to the subunit; and
n is an integer greater than or equal to zero; optionally n is an integer in the range of 0 to 10; optionally n is an integer in the range of 0 to 10; optionally n is an integer in the range of 1 to 4; optionally, n is 1, 2, 3 or 4.

101. The multi-conjugate of claim 100, wherein at least one of the subunits ******** present in Structure 3 is not an oligonucleotide.

102. The multi-conjugate of claim 100 or 101, wherein at least one of the subunits ******** present in Structure 3 comprises an oligopeptide or a protein.

103. The multi-conjugate of claim 100, wherein at least one functional moiety is present.

104. The multi-conjugate of claim 103, wherein the at least one functional moiety that is present is a targeting ligand.

105-116. (canceled)

117. A composition comprising a multimeric oligonucleotide of claim 1 and a pharmaceutically acceptable excipient.

118-126. (canceled)
